(19) United States
(12) Patent Application Publication
(10) Pub. No.: US 2015/0252319 A1
(43) Pub. Date: Sep. 10, 2015

(54) pH CONTROLLED YEAST PROPAGATION

(71) Applicant: DSM IP ASSETS B.V., Heerlen (NL)

(72) Inventors: Hans Marinus Charles Johannes De Bruijn, Echt (NL); Paul Klaassen, Echt (NL)

(21) Appl. No.: 14/439,275

(22) PCT Filed: Nov. 1, 2013
(86) PCT No.: PCT/EP2013/072871

§ 371 Date: Apr. 29, 2015

(30) Foreign Application Priority Data
Nov. 7, 2012 (EP) 12191660.5

(51) Int. Cl. C12N 1/16; C12P 7/06; C12N 1/22

(52) U.S. Cl. CPC C12N 1/16; C12N 1/22; C12P 7/06

(57) ABSTRACT

The invention relates to a process for the aerobic propagation of yeast wherein the yeast is grown in a reactor, comprising the following steps:
a) filling the reactor with carbon source and an initial yeast population,
b) optionally growing the initial yeast population in the reactor in batch mode,
c) measuring the pH in the reactor,
d) adding lignocellulosic hydrolysate to the reactor in fed batch mode at a rate to set the pH in the reactor at a predetermined value,
and
e) after sufficient propagation, isolation of yeast from the reactor.

The invention further relates to yeast propagated according to that propagation process and to a process for the production of fermentation product wherein sugar comprising hexose and pentose is anaerobically fermented to fermentation product with the propagated yeast.

### FIELD OF THE INVENTION

The invention is directed to a propagation process for yeast. In particular the invention relates to a propagation process wherein yeast is propagated on lignocellulosic hydrolysate.

### BACKGROUND OF THE INVENTION

There are nowadays processes proposed to use lignocellulosic material as a source for the production of fuel and of base chemicals. They are aimed at commercially-viable production of these products from lignocellulosic feedstocks.

In such processes lignocellulosic material may for example be pretreated, then hydrolysed and subsequently the resulting hydrolysate that comprises hexose and/or pentose sugar may be converted by yeast into fermentation product. These processes may take place in a large scale Integrated Bioprocess Facility (IBF). The yeast fermentation is usually conducted under anaerobic conditions in the fermentation part of the IBF.

To be able to supply enough yeast to the fermentation, yeast is propagated either in the IBF or elsewhere and shipped to the IBF. Propagation is usually conducted under aerobic conditions.

From German patent 300662, there is known a process for the aerobic propagation of yeast wherein the propagation is started with broth that is strongly diluted and then undiluted broth is added slowly. The part of the process of slow addition of broth is herein called fed-batch phase of the process. The overall process including a fed-batch phase is herein called fed-batch process. The advantage of the known fed-batch process is that excessive formation of ethanol is avoided and that larger broth concentrations than in diluted batch process can be used.

From Kollaras, A. et al, Ethanol Producer Magazine, August 2012, page 52-54 there is known aerobic propagation of yeast (S. cerevisiae) on xylose containing stillage and it is described that: “Within a submerged aerobic propagator similar to that in which baker's yeast is grown, S. cerevisiae MBG 3248 converted acetic acid, lactic acid, ethanol, glycerol, residual six carbon sugars and xylose into yeast biomass at an observed yield of 0.35 g of yeast per gram of total usable carbon”. Disadvantage of this known process is that since xylose rich stillage is used for biomass formation, excess yeast (129,000 tons of feed yeast) is produced. The xylose converted in feed yeast can better be used to produce fermentation product in the IBF. Disadvantage to all currently known propagation process on acidic lignocellulosic hydrolysate is that yeast growth is inhibited by acetic acid and/or sugar degradation products. Nevertheless such hydrolysate may be present and available from the IBF, and is cheaper than conventional propagation carbon sources, and thus would be a desirable carbon source.

### SUMMARY OF THE INVENTION

An object of the invention is to provide a propagation process wherein lignocellulosic hydrolysate may be used as carbon source. A further object is to provide a propagation process that may be operated in a stable fashion. Another object is to provide a propagation process that may be performed in multiple cycles, wherein part of the propagation mixture is used for the next round of propagation. A further object is to provide a propagation process that avoids excess production of yeast. One or more of these objects are attained according to the invention.

According to the present invention, there is provided process for the aerobic propagation of yeast wherein the yeast is grown in a reactor comprising the following steps:
a) filling the reactor with carbon source and an initial yeast population,
b) optionally growing the initial yeast population in the reactor in batch mode,
c) measuring the pH in the reactor,
d) propagation while adding lignocellulosic hydrolysate to the reactor in fed batch mode at a rate to set the pH in the reactor at a predetermined value
and
e) after sufficient propagation, isolation of yeast from the reactor.

According to the invention a propagation process is obtained a wherein lignocellulosic hydrolysate may be used as carbon source, that may be operated in a stable fashion, in multiple cycles and that avoids excess production of yeast.

A further advantage of the propagation according to the invention is that the yeast is adapted during propagation in one or more cycles on the hydrolysate in such a way that its performance in ethanol fermentation of lignocellulosic hydrolysate is increased compared to yeast not propagated according to the invention on hydrolysate.

The invention also provides:
yeast produced according to the above propagation process, and
a process for producing a fermentation product which uses the yeast according to the above propagation process, in particular where the fermentation product is ethanol.

### BRIEF DESCRIPTION OF THE DRAWINGS

FIG. 1: Sugar consumption and ethanol production during yeast propagation against time (h). The solid squares are measurements of sugar concentration (g/l). Open circles ethanol concentration (g/L). See legend.

FIG. 2: Biomass formation and inhibitor concentrations against time (h). Triangles indicate inhibitor concentrations (g/l); with solid line representing acetic acid concentrations. Open circle with solid line indicates biomass (yeast) concentration (as dry biomass) (g/l) calculated from OD700. Glycerol concentration is indicated as open circle with intermitted line. See legend.

FIG. 3: Propagator parameters against time (h). Oxygen concentration (pO2(%) -.-, pH-- and temperature (° C.). See legend.

FIG. 4: Yeast growth. Shown is (In mass(x)) against time (h). The straight part of curve indicated exponential growth. See legend.

### DETAILED DESCRIPTION OF THE INVENTION

Throughout the present specification and the accompanying claims the words “comprise” and “include” and variations such as “comprises”, “comprising”, “includes” and “including” are to be interpreted inclusively. That is, these words are intended to convey the possible inclusion of other elements or integers not specifically recited, where the context allows.

Herein reactor, propagator or fermentor may be used for the reactor in which propagation can take place. Propagation is herein aerobic fermentation with the aim to increase a yeast population. Ethanol fermentation herein relates to fermentation with the aim to produce ethanol and is usually conducted anaerobic. The reactor in which ethanol fermentation occurs is herein ethanol fermentor or ethanol reactor.

According to the invention, the aerobic propagation of yeast wherein the yeast is grown in a reactor comprises the following steps:
a) filling the reactor with carbon source and an initial yeast population,
b) optionally growing the initial yeast population in the reactor in batch mode,
c) measuring the pH in the reactor,
d) propagation while adding lignocellulosic hydrolysate to the reactor in fed-batch mode at a rate to maintain the pH at a predetermined level,
and
e) after sufficient propagation, isolation of yeast from the reactor.

In this process steps a), b) and e) may be conducted in conventional way, though some parameters of these steps may be different then in the specific known conventional processes as in described in more detail below. In step a) any suitable carbon source may be used. In an embodiment, in step a), the carbon source is diluted lignocellulosic hydrolysate, more specifically lignocellulosic hydrolysate that is two or more fold diluted in water.

In step c) measuring of pH in the propagation reactor (also herein called propagator) may be conducted with any conventional pH measurement instument, such as an industrially used pH probe or industrially used pH controller. The pH value is used to trigger the start of feed of lignocellulosic hydrolysate, i.e. the start of the fed-batch phase of the propagation. In step c) the signal from the pH probe or controller may be used to open a metering valve that is connected a to feed line for the lignocellulosic hydrolysate.

In step d) the fed batch phase, the pH probe signal or pH controller signal is used to provide a controlled feed rate of lignocellulosic hydrolysate though the metering valve into the reactor, in a way that the pH is set the pH in the reactor at a predetermined value. The predetermined value may be a single pH value or may change over time if a pH profile is set. In an embodiment the pH predetermined value is substantially constant. In such embodiment, the pH in the propagator in the fed-batch phase is from pH 4 to 10 or from pH 4 to pH 7. In an embodiment, the lignocelluloic hydrolysate is fed at a rate that the pH of the mixture in the propagator remains higher than the pH of the lignocellulosic hydrolysate that is fed into the propagator.

The lignocellulosic hydrolysate may be acidic. In an embodiment, the lignocellulosic hydrolysate comprises organic acid. Examples of organic acids possible in lignocellulosic hydrolysate are acetic acid and formic acid. In an embodiment the organic acid is acetic acid. Acidic lignocellulosic hydrolysate is common product from pretreatment wherein acid is used, which results in formation of acetic acid.

Accordingly in step d) during fed-batch propagation carbon source and optionally other ingredients as phosphoric acid, ammonia and minerals are fed to the yeast in the propagator at a pH controlled rate. This rate is designed to feed just enough sugar and nutrients to the yeast to maximize multiplication and at the same time just low enough to prevent the production of alcohol, and all or most of the acetic acid and/or other acidic inhibitors from the feed is consumed.

In an embodiment, the yeast consumes xylose in the lignocelluloic hydrolysate, preferably substantially all xylose.

In an embodiment, in the process no base needs to be added to the mixture in the reactor. In an embodiment during the propagation the acetic acid concentration (g/L) is 0.5 g/L or less, preferably 0.2 g/L or less.

In an embodiment, the propagation is conducted until at least five generations of growth of the yeast population are realized. In an embodiment the propagation is conducted until growth of the yeast population for 5 to 6 generations compared to the initial yeast population. In an embodiment the batch phase of propagation is conducted until growth of the yeast population for two generations and the fed batch phase for three or more generations. A generation of growth herein means a doubling of yeast biomass in weight (g).

### Exponential Growth in Batch Cultures

The definition of a generation here is a doubling of yeast biomass. The doubling of the amount of biomass can be described by Cx (biomass concentration) at given time to be given by the following equation:

Cx(t)=Cx(0)*e^{(μ*t)} (eq. 1)

The doubling time (Td in h) or generation time (Tg h) can be derived from the is equation by substituting Cx(t)=2*Cx(0).

Td=LN(2)/μ(hr) (eq. 2)

Where μ=specific growth rate in g biomass/g biomass/h or 1/h).

The biomass growth rate can be measured by various means: The increase of biomass amount can be analyzed by determining the amount of cells per weight or volume unit of a culture using any of the following method or a suitable alternative method:
Turbidity
Optical Density in the visible light spectrum (usual range: 600 nm to 700 nm) of a culture
A pellet volume after centrifugation,
The dry weight content after drying at constant weight at 105° C.
Cell count per volume (microscopically),
Colony Forming Unit (CFU/ml) after plating on a solid agar medium and growing colonies on a plate from single cells

Alternatively one can derive the amount of biomass from a metabolic activity measured in a closed reactor system such as:
The rate of carbondioxide production (CPR carbondioxide production rate or CER Carbon Dioxide Evolution Rate generally expressed as mmol CO2/L/hr)
The rate of oxygen consumption (OUR Oxygen Uptake Rate mmol O2/L.hr)
Substrate uptake rate (rs=substrate uptake rate in g /L.hr uptake rate of glucose, xylose, arabinose or ammonia)

When Ln(Cx) or LN (CPR), LN(OUR) or LN (rs) or is plotted versus time in an exponential growth experiment (no nutrient limitations and no toxic products formed) a straight is obtained with the slope being the specific growth rate μ. With μ and eq. 2 one can calculate the doubling time and with the growth time one can calculate the amount of doublings or the number of generations.

### Non Exponential Growth

In non-exponential growth experiments, e.g. a fed batch fermentation with constant feed or a continuous fermentation, the amount of generations is determined by calculating

Mx=Cx*Volume (biomass conc. in g/L*liter of broth produced in gr biomass) (eq 3.)

yielding the total mass of yeast biomass in g dry matter of total CFU (=CFU/ml*ml of culture produced, or OD*vol.

A factor two increase in Mx means one generation.

The principal of the Non-exponential growth is also applicable to the exponential growth systems as described above.

In step e) after sufficient propagation, yeast may be isolated from the reactor or fed as a whole broth to an ethanol fermentation reactor. These steps may be executed in conventional way. In an embodiment, part of the propagated yeast is recycled to the propagator.

### Yeast Population

The initial yeast population should have an appropriate size that is dependent on the size of the propagation reactor and the available amount of carbon source in the reactor. In an embodiment the initial yeast population may originate from a pure culture tube or frozen vial of the appropriate yeast strain. The pure culture tube or frozen vial may be used as inoculum for a pre-pure culture tank, a small pressure vessel where seed is grown in medium under strict sterile conditions. Following growth, the contents of this vessel are transferred to a larger pure culture reactor where propagation is carried out with some aeration, again under sterile conditions. From the pure culture vessel, the grown cells are transferred to a series of progressively seed and semi-seed propagators. These early stages are conducted as batch fermentations.

In an embodiment yeast is capable of metabolizing organic acid, preferably of metabolizing acetic acid. We have found that yeast, in particular S. cerevisiae can consume acetic acid and other organic acids if present in low concentration, such as for instance 5 g/l or less, 4 g/l or less, 3 g/l or less, 2 g/l or less, 1 g/l or less, or 0.5 g/l or less and only when sugars and other carbon sources have been depleted.

The yeast used in the propagation process as initial yeast population may be (genetically engineered) yeast. Genetic engineering is hereinafter described in more detail. Yeasts are herein defined as eukaryotic microorganisms and include all species of the subdivision Eumycotina (Alexopoulos, C. J., 1962, In: Introductory Mycology, John Wiley & Sons, Inc. , New York) that predominantly grow in unicellular form.

Yeasts may either grow by budding of a unicellular thallus or may grow by fission of the organism. A preferred yeast as a yeast may belong to the genera Saccharomyces, Kluyveromyces, Candida, Pichia, Schizosaccharomyces, Hansenula, Kloeckera, Schwanniomyces or Yarrowia. Preferably the yeast is one capable of anaerobic fermentation, more preferably one capable of anaerobic alcoholic fermentation. In an embodiment the yeast is Saccharomyces cerevisiae.

In an embodiment, the yeast is an industrial yeast. An industrial yeast cell may be defined as follows. The living environments of yeast cells in industrial processes are significantly different from that in the laboratory. Industrial yeast cells must be able to perform well under multiple environmental conditions which may vary during the process. Such variations include change in nutrient sources, pH, ethanol concentration, temperature, oxygen concentration, etc., which together have potential impact on the cellular growth and ethanol production of Saccharomyces cerevisiae. Under adverse industrial conditions, the environmental tolerant strains should allow robust growth and production. Industrial yeast strains are generally more robust towards these changes in environmental conditions which may occur in the applications they are used, such as in the baking industry, brewing industry, wine making and the ethanol industry. Examples of industrial yeast (S. cerevisiae) are genetically engineered Ethanol Red® (Fermentis) Fermiol® (DSM) and Thermosacc® (Lallemand). In an embodiment the yeast is inhibitor tolerant. Inhibitor tolerant yeast cells may be selected by screening strains for growth on inhibitors containing materials, such as illustrated in Kadar et al, Appl. Biochem. Biotechnol. (2007), Vol. 136-140, page 847-858, wherein an inhibitor tolerant S. cerevisiae strain ATCC 26602 was selected. RN1016 is a xylose and glucose fermenting S. cerevisiae strain from DSM, Bergen op Zoom, the Netherlands.

In an embodiment the yeast is capable of converting hexose (C6) sugars and pentose (C5) sugars. In an embodiment the yeast can an-aerobically ferment at least one C6 sugar and at least one C5 sugar. For example the yeast is capable of using L-arabinose and xylose in addition to glucose an-aerobically. In an embodiment, the yeast is capable of converting L-arabinose into L-ribulose and/or xylulose 5-phosphate and/or into a desired fermentation product, for example into ethanol. Organisms, for example S. cerevisiae strains, able to produce ethanol from L-arabinose may be produced by modifying a host yeast introducing the araA (L-arabinose isomerase), araB (L-ribuloglyoxalate) and araD (L-ribulose-5-P4-epimerase) genes from a suitable source. Such genes may be introduced into a host cell in order that it is capable of using arabinose. Such an approach is given is described in WO2003/095627. araA, araB and araD genes from Lactobacillus plantarum may be used and are disclosed in WO2008/041840. The araA gene from Bacillus subtilis and the araB and araD genes from Escherichia coli may be used and are disclosed in EP1499708. In another embodiment, araA, araB and araD genes may derived from of at least one of the genus Clavibacter, Arthrobacter and/or Gramella, in particular one of Clavibacter michiganensis, Arthrobacter aurescens, and/or Gramella forsetii, as disclosed in WO 2009011591. In an embodiment, the yeast may also comprise one or more copies of xylose isomerase gene and/or one or more copies of xylose reductase and/or xylitol dehydrogenase.

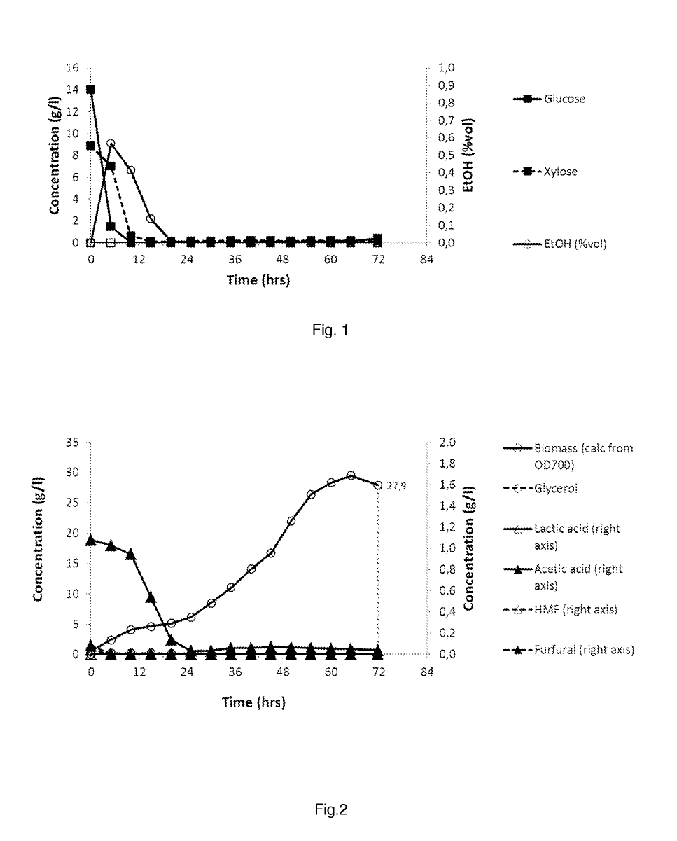

The yeast may comprise one or more genetic modifications to allow the yeast to ferment xylose. Examples of genetic modifications are introduction of one or more xylA-gene, XYL1 gene and XYL2 gene and/or XKS1-gene; deletion of the aldose reductase (GRE3) gene; overexpression of PPP-genes TAD, TKL1, RPE1 and RKI1 to allow the increase of the flux through the pentose phosphate pathway in the cell. Examples of genetically engineered yeast is described in EP1468093 and/or WO2006009434. As shown in FIG. 1, such yeast displays a specific preference in c-source utilization; first glucose is taken up and converted to yeast biomass and co-products, most notably EtOH (due to the Crabtree effect). As the glucose concentration decreases, pentose (xylose) is consumed, at which point EtOH production ceases due to a decrease of glycolytic flux (no more overflow metabolism). During the later phase of xylose utilization, the previously produced EtOH, as well as acetic acid are metabolized, the latter causing a rise of the pH of the fermentation broth. By feeding undiluted, acidic hydrolysate, the pH is fixed at a certain point along this pH slope, effectively keeping the acetic acid concentration during the feed-phase lower than in both the batch-phase and the undiluted hydrolysate feed.

As the consumption of acetic acid, which causes the increase in pH, occurs after, or at least partly overlaps consumption of both xylose and EtOH, a propagation process using this feeding strategy intentionally and inevitably results in a broth with depleted, or at least strongly decreased pentose concentrations. These pentoses are also converted to yeast biomass, increasing total yeast biomass concentration in the broth, allowing for smaller installed aerated fermentation volume (capex). This is fundamentally different from WO2011/022840 (Geertman), in which enrichment of the xylose/glucose ratio is pursued by converting hexoses while minimizing pentose conversion. To further illustrate this fundamental difference, using a strain as described above in combination with the latter strategy would lead to very limited-to no detoxification by acetic acid conversion, as pentose conversion strongly overlaps pentose uptake in such a strain. As the described feeding strategy is aimed at abolishing, or at least minimizing acetic acid concentration (and thereby inhibition of the propagated yeast), it is has benefits over controlling/limiting a feed of undiluted hydrolysate by on-line EtOH measurement (Petersson et. al. 2006, Andreas et. al. 2007), in that in the latter the undiluted hydrolysate is fed before the yeast metabolizes the acetic acid in the broth which is therefore not continually consumed, therefore lacks the detoxifying effect, and still suffers severely from growth inhibition of the yeast, especially at acetic acid concentrations common in industrial hydrolysates (≧5 g/l). In the Integrated Bioprocess Facility, the fermentation product of the propagated yeast herein may be any useful product. In one embodiment, it is a product selected from the group consisting of ethanol, n-butanol, isobutanol, lactic acid, 3-hydroxy-propionic acid, acrylic acid, acetic acid, succinic acid, fumaric acid, malic acid, itaconic acid, maleic acid, citric acid, adipic acid, an amino acid, such as lysine, methionine, tryptophan, threonine, and aspartic acid, 1,3-propane-diol, ethylene, glycerol, a β-lactam antibiotic and a cephalosporin, vitamins, pharmaceuticals, animal feed supplements, specialty chemicals, chemical feedstocks, plastics, solvents, fuels, including biofuels and biogas or organic polymers, and an industrial enzyme, such as a protease, a cellulase, an amylase, a glucanase, a lactase, a lipase, a lyase, an oxidoreductases, a transferase or a xylanase. For example the fermentation products may be produced by yeast propagated accoding to the invention, following prior art cell preparation methods and fermentation processes, which examples however should herein not be construed as limiting. n-butanol may be produced by cells as described in WO2008121701 or WO2008086124; lactic acid as described in US2011053231 or US2010137551; 3-hydroxy-propionic acid as described in WO2010010291; acrylic acid as described in WO2009153047.

For the recovery of the fermentation product in the Integrated Bioprocess Facility existing technologies are used. For different fermentation products different recovery processes are appropriate. Existing methods of recovering ethanol from aqueous mixtures commonly use fractionation and adsorption techniques. For example, a beer still can be used to process a fermented product, which contains ethanol in an aqueous mixture, to produce an enriched ethanol-containing mixture that is then subjected to fractionation (e.g., fractional distillation or other like techniques). Next, the fractions containing the highest concentrations of ethanol can be passed through an adsorber to remove most, if not all, of the remaining water from the ethanol.

### Propagation

Propagation is herein any process of yeast growth that leads to increase of an initial yeast population. Main purpose of propagation is to increase a yeast population using the yeast's natural reproduction capabilities as living organisms. There may be other reasons for propagation, for instance, in case dry yeast is used, propagation is used to rehydrate and condition the yeast, before it is grown. Fresh yeast, whether active dried yeast or wet cake may be added to start the propagation directly.

The conditions of propagation are critical for optimal yeast production and subsequent fermentation, such as for example fermentation of lignocellulosic hydrolysate into ethanol. They include adequate carbon source, aeration, temperature and nutrient additions. Tank size for propagation and is normally between 2 percent and 5 percent of the (lignocellulosic hydrolysate to ethanol) fermentor size.

First, the yeast needs a source of carbon. The source of carbon is herein in the fed batch phase lignocellulosic hydrolysate. The carbon source is needed for cell wall biosynthesis and protein and energy production.

For the batch phase, the carbon source may be diluted lignocellulosic hydrolysate. Dilution (with water) is advantageous since if the yeast is propagated on undiluted lignocellulosic, since it commonly contains a too high level of inhibitors, so it is poisonous for the yeast. This means that with propagation will proceed very slow, the number of generations possible is about two at most and the propagated yeast will have bad fermentation behaviour. The dilution factor may be determined by the skilled person based on the sugar content and the level of inhibitors of the lignocellulosic hydrolysate. In an embodiment, the carbon source is lignocellulosic hydrolysate that is two or more fold diluted in water, more than threefold diluted, more than fourfold diluted, more than fivefold diluted or 6, 7, 8 9, 10, 15 or 20-fold diluted. In the batch phase also other carbon sources than diluted lignocellulosic hydrolysate may be used. The carbon source may be any form of sugar, for instance glucose, and the sugar may be in any form such as crystallized or in less pure form as for instance melasse.

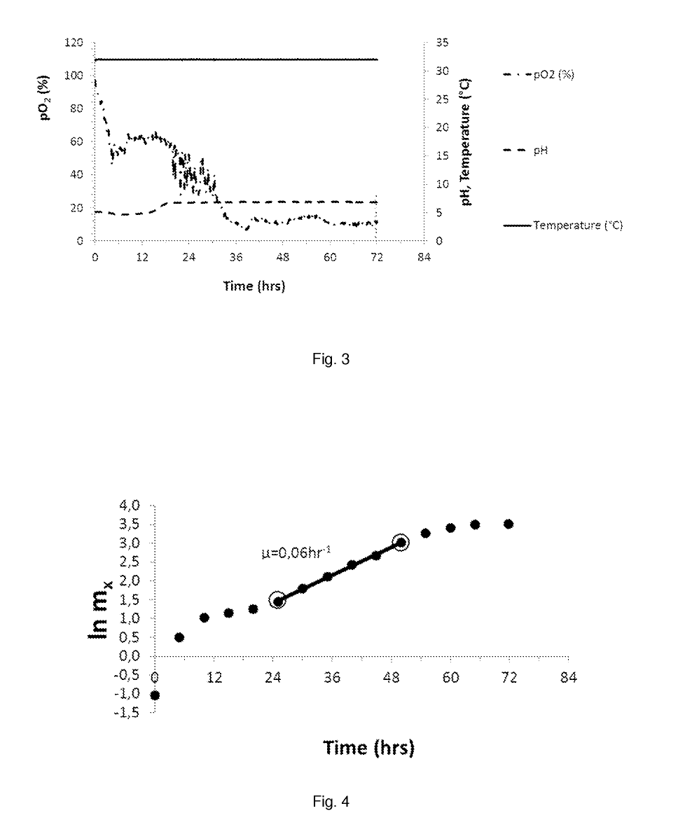

In an embodiment, in the batch phase, sugar levels are targeted at or just above 2 percent (w/w) at the beginning of propagation. Since this concentration is higher than that which causes Crabtree effect, accordingly ethanol is produced, see example 1, FIG. 1, where initially the ethanol concentration increases. However, we found that this ethanol is subsequently, after sugars and glycerol are depleted, is consumed by the yeast. Further we found that ethanol and acetic acid and other acids are then consumed (see example 1, FIG. 2). After the acetic acid and/or other acids are consumed, the pH in the propagator will rise. This pH rise is used in the invention.

In addition to a carbon source, additional nutrients above what is naturally provided in the lignocellulosic hydrolysate may be added to optimize growth. Nitrogen, e.g. in the form of urea is most often used at a rate of between 300 parts per million to 500 parts per million or higher. Although ammonia is also a good nitrogen source for the yeast, it can be inhibitory to yeast during rehydration. Failure to add additional nitrogen can cause sluggish yeast growth, resulting in abnormally low yeast counts or slower metabolism. Additional ingredients like magnesium and zinc are sometimes added for additional benefit.

Propagation is an aerobic process, thus the propagation tank must be properly aerated to maintain a certain level of dissolved oxygen. Adequate aeration is commonly achieved by air inductors installed on the piping going into the propagation tank that pull air into the propagation mix as the tank fills and during recirculation. The capacity for the propagation mix to retain dissolved oxygen is a function of the amount of air added and the consistency of the mix, which is why water is often added at a ratio of between 50:50 to 90:10 mash to water. “Thick” propagation mixes (80:20 mash-to-water ratio and higher) often require the addition of compressed air to make up for the lowered capacity for retaining dissolved oxygen. The amount of dissolved oxygen in the propagation mix is also a function of bubble size, so some ethanol plants add air through spargers that produce smaller bubbles compared to air inductors. Along with lower glucose, adequate aeration is important to promote aerobic respiration, which differs from the comparably anaerobic environment of fermentation. One sign of inadequate aeration or high glucose concentrations is increased ethanol production in the propagation tank.

Generally during propagation, yeast requires a comfortable temperature for growth and metabolism, for instance the temperature in the propagation reactor is between 25-40 degrees Celcius. Generally lower temperatures result in slower metabolism and reduced reproduction, while higher temperatures can cause production of stress compounds and reduced reproduction. In an embodiment the propagation tanks are indoors and protected from the insult of high summer or low winter temperatures, so that maintaining optimum temperatures of between within the range of 30-35 degrees C. is usually not a problem.

Another common question is how long to propagate yeast before adding it to the propagator. Propagation times vary between plants, but most often range between six and 100 hours. An indication may be the time it takes for the yeast to reach exponential growth phase. Longer propagation cycles can result in the yeast entering stationary phase or a stage of decline due to depletion of nutrients and accumulation of byproducts such as acetic acid, which can cause a subsequent lag in yeast performance once in the propagator.

Shorter propagation cycles do not allow time for adequate doubling or reproduction of the yeast, one of the primary reasons for propagating in the first place. Determining optimal drop times for propagation may involve charting growth under the conditions described above and deciding when the yeast has reached exponential growth in relation to when it enters into the subsequent stationary or rapid decline phases.

Bacterial or wild yeast contamination is rarely a problem during propagation because yeast propagation tanks are smaller and can be more easily cleaned than fermentation tanks. Apart from cleaning, antibacterial products may be added to prevent growth of unwanted microbes.

In summary, yeast propagation is an integral part of the fuel ethanol production process. By following the aforementioned guidelines, propagation can be optimized and problems in fermentation minimized.

During fed-batch propagation carbon source and optionally other ingredients as phosphoric acid, ammonia and minerals are fed to the yeast in the propagator at a controlled rate. This rate is designed to feed just enough sugar and nutrients to the yeast to maximize multiplication and prevent the production of alcohol. In an embodiment, the rate of lignocellulosic hydrolysate fed into the fed batch reactor is 0.10 h⁻¹ or less or from 0.01 h⁻¹ to 0.10 h⁻¹.

In an embodiment, the fed-batch fermentations are not completely sterile. It is not economical to use pressurized tanks to guarantee sterility of the large volumes of air required in these fermentors (propagators) or to achieve sterile conditions during all the transfers through the many pipes, pumps and centrifuges. Extensive cleaning of the equipment, steaming of pipes and tanks and filtering of the air is practiced to insure as aseptic conditions as possible.

At the end of the semi-seed propagation, the contents of the vessel are pumped to a series of separators that separate the yeast from the spent hydrolysate. Alternatively a whole propagator broth may be pumped and added to the commercial propagators, optionally after storage in a buffer tank.

Commercial propagations may be carried out in large fermentors (propagators) with working volumes up to 50,000 gallons or more. To start the commercial propagation, a volume of water, referred to as set water, is pumped into the propagator. Next, in a process referred to as pitching, yeast from semi-feed propagation or from a storage tank is transferred into the fermentor. Following addition of the seed yeast, aeration, cooling and nutrient additions are started to begin the fermentation. At the start of the fermentation, the liquid seed yeast and additional water may occupy only about one-third to one-half of the fermentor volume. Constant additions of nutrients during the course of fermentation bring the fermentor to its final volume. The rate of nutrient addition increases throughout the fermentation because more nutrients have to be supplied to support growth of the increasing cell population. The number of yeast cells increase about one to two times, two to three times, three to four times, four to five times, five to six times, five to seven times or five to eight times during this propagation.

Air is provided to the fermentor (propagator) through a series of perforated tubes located at the bottom of the vessel. The rate of airflow is about one volume of air per fermentor volume per minute. A large amount of heat is generated during yeast growth and cooling is accomplished by internal cooling coils or by pumping the fermentation liquid, also known as broth, through an external heat exchanger. The addition of nutrients and regulation of pH, temperature and airflow are carefully monitored and controlled by computer systems during the entire production process.

At the end of fermentation, the fermentor broth is separated by nozzle-type centrifuges, washed with water and re-centrifuged to yield a yeast cream with a solids concentration of approximately 18%. The yeast cream is cooled to about 45 degrees Fahrenheit and may be stored in a separate, refrigerated stainless steel cream tank or use directly in the main fermentations of the Integrated Bioprocess Facility. Alternatively cream yeast can be loaded directly into tanker trucks and delivered to customers equipped with an appropriate cream yeast handling system. Alternatively, the yeast cream can be pumped to a plate and frame filter press and dewatered to a cake-like consistency with a 30-32% yeast solids content. This press cake yeast is crumbled into pieces and packed into 50-pound bags that are stacked on a pallet. The yeast heats up during the pressing and packaging operations and the bags of crumbled yeast must be cooled in a refrigerator for a period of time with adequate ventilation and placement of pallets to permit free access to the cooling air. Palletized bags of crumbled yeast are then distributed to customers in refrigerated trucks.

Alternatively in an IBF, a whole propagator broth may be pumped and added to the ethanol fermentation vessels in the IBF, optionally after storage in a buffer tank.

### Lignocellulose Hydolysate

Lignocellulosic hydrolysate is herein any hydrolysed lignocellulose. Lignocelllulose is herein biomass. It herein includes hemicellulose and hemicellulose parts of biomass. Also lignocellulose includes lignocellulosic fractions of biomass. Suitable lignocellulosic materials may be found in the following list: orchard primings, chaparral, mill waste, urban wood waste, municipal waste, logging waste, forest thinnings, short-rotation woody crops, industrial waste, wheat straw, oat straw, rice straw, barley straw, rye straw, flax straw, soy hulls, rice hulls, rice straw, corn gluten feed, oat hulls, sugar cane, corn stover, corn stalks, corn cobs, corn husks, switch grass, miscanthus, sweet sorghum, canola stems, soybean stems, prairie grass, gamagrass, foxtail; sugar beet pulp, citrus fruit pulp, seed hulls, cellulosic animal wastes, lawn clippings, cotton, seaweed, trees, softwood, hardwood, poplar, pine, shrubs, grasses, wheat, wheat straw, sugar cane bagasse, corn, corn husks, corn hobs, corn kernel, fiber from kernels, products and by-products from wet or dry milling of grains, municipal solid waste, waste paper, yard waste, herbaceous material, agricultural residues, forestry residues, municipal solid waste, waste paper, pulp, paper mill residues, branches, bushes, canes, corn, corn husks, an energy crop, forest, a fruit, a flower, a grain, a grass, a herbaceous crop, a leaf, bark, a needle, a log, a root, a sapling, a shrub, switch grass, a tree, a vegetable, fruit peel, a vine, sugar beet pulp, wheat midlings, oat hulls, hard or soft wood, organic waste material generated from an agricultural process, forestry wood waste, or a combination of any two or more thereof.

An overview of some suitable sugar compositions derived from lignocellulose and the sugar composition of their hydrolysates is given in table 1. The listed lignocelluloses include: corn cobs, corn fiber, rice hulls, melon shells, sugar beet pulp, wheat straw, sugar cane bagasse, wood, grass and olive pressings.

The following Examples illustrate the invention.

### EXAMPLES

### Example 1

In example 1, as lignocellulosic hydrolysate, enzymatically hydrolyzed pretreated corn stover (17% dry matter) was used. The composition of the hydrolysate is given in table 1.

TABLE 1
Composition of the lignocellulosic hydrolysate
(HPLC (H-column) analysis)

| | | |
|---|---|---|
| Glucose | (g/l) | 69.8 |
| Xylose | (g/l) | 43.4 |
| Glycerol | (g/l) | 0.2 |
| Formic acid | (g/l) | 0.2 |
| Acetic acid | (g/l) | 5.1 |
| Ethanol | (% vol) | 0 |
| HMF | (g/l) | 0.19 |
| Furfural | (g/l) | 0.98 |
| Arabinose | (g/l) | 5.2 |

### Fermentation Parameters

A fed batch propagation reactor (1500 ml ) was filled with 709 g 5 times diluted lignocellulosic hydrolysate. There was added 0.2 g/l MgSO4, 1.1 g/l (NH4)2SO4, 4.5 g/l urea, 4 ml/l vitamin solution and 4 ml/l trace elements (As in Verduyn et al, 1992, ref. see below). The pH was adjusted to 5 with NH4OH. Temperature was of the fed batch reactor was controlled at 32° C. Dissolved oxygen levels were kept above 9% by aeration at 3 vvm (final volume) in combination with a stirring cascade (controlled between 200-700 rpm). Starting volume of the propagation experiment was 700 ml. pH of the broth was controlled at 6.8 by adding additional whole (not-diluted) cellulosic hydrolysate. Final volume of the propagation experiment was 1190 ml.

### Propagation

The fed-batch reactor was filled with 700 ml 5× diluted hydrolysate (in demineralized water) and inoculated to 0.35 g/l (dry yeast biomass) RN1016.

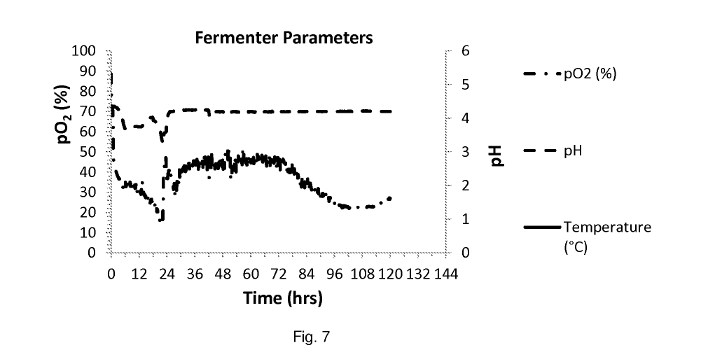

The results of the propagation fermentation are shown in FIGS. 1 to 4.

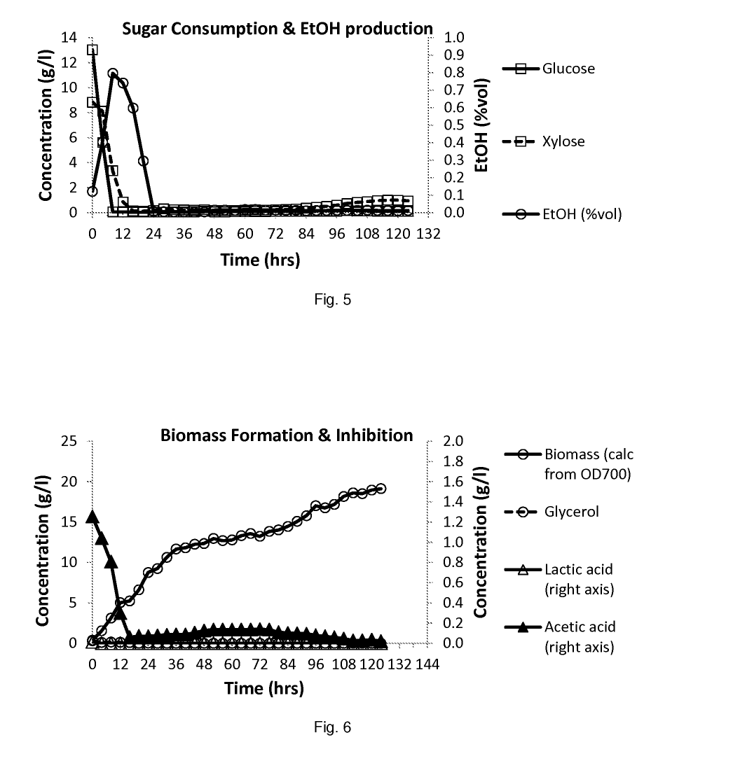

From FIG. 1 it is clear that after 24 hrs all carbon sources, including most of the acetic acid (leaving ˜0.03 g/l) were consumed, the latter causing a pH increase from 5 (start) to 6.8 at which point the pH control (feed) was triggered, which maintained the pH constant at 6.8 by adding increasing amounts of hydrolysate feed. Yeast biomass concentration increased with a maximum growth rate of 0.06 hr⁻¹ to approximately 28 g/l⁻¹ corresponding to a biomass yield of 0.42 g*g⁻¹ per consumed sugar. From FIG. 2 it is clear that yeast propagation proceeds until about 72 h. FIG. 3 gives the pH profile, it can be seen that pH is kept substantially constant by addition of lignocellulosic hydrolysate from 36 h onwards. In FIG. 3 it is shown that from 24 h till about 50 h exponential growth occurs.

The example shows that propagation on lignocellulosic hydrolysate is possible and can be stable executed. Since lignocellulosic hydrolysate is used, the amount of yeast that is produced can be in any desirable amount, so no excess yeast is produced. Further the propagated yeast may be recycled and used for a new batch of propagation.

### Example 2

In example 2, as lignocellulosic hydrolysate, enzymatically hydrolyzed pretreated corn stover (17% dry matter) was used. The composition of the hydrolysate is given in table 2.

TABLE 2
Composition of the lignocellulosic hydrolysate
(HPLC (H-column) analysis)

| | | |
|---|---|---|
| Glucose | (g/l) | 68.2 |
| Xylose | (g/l) | 44.8 |
| Glycerol | (g/l) | 0.0 |
| Formic acid | (g/l) | 0.3 |
| Acetic acid | (g/l) | 5.2 |
| Ethanol | (% vol) | 0 |
| HMF | (g/l) | 0.18 |
| Furfural | (g/l) | 1.02 |
| Arabinose | (g/l) | 5.2 |

### Fermentation Parameters

A fed batch propagation reactor (1500 ml) was filled with 709 g 5 times diluted lignocellulosic hydrolysate. There was added 0.2 g/l MgSO4, 1.1 g/l KH2PO4, 4.5 g/l urea, 4 ml/l vitamin solution and 4 ml/l trace elements (As in Verduyn et al, 1992, ref. see below). The pH was of the hydrolysate (4.3) was not adjusted after enzymatic hydrolysis. Temperature was of the fed batch reactor was controlled at 32° C. Dissolved oxygen levels were kept above 9% by aeration at 3 vvm (final volume) in combination with a stirring cascade (controlled between 200-700 rpm). Starting volume of the propagation experiment was 600 ml. pH of the broth was controlled at 4.2 by adding additional whole (not-diluted) cellulosic hydrolysate. Final volume of the propagation experiment was 1600 ml.

### Propagation

The fed-batch reactor was filled with 700 ml 5× diluted hydrolysate (in demineralized water) and inoculated to 0.39 g/l (dry yeast biomass) RN1016.

The results of the propagation fermentation are shown in FIGS. 5 to 7.

From FIGS. 5 and 6 it is clear that after 16 hrs all carbon sources, including most of the acetic acid (leaving 0.1 g/l) were consumed, the latter causing a pH increase from 3.7 (broth pH decreased from 4.3 (start) to 3.7 in the glucose-phase) to 4.2 at which point the pH control (feed) was triggered, which maintained the pH constant at 4.2 by adding increasing amounts of hydrolysate feed. The dip at 24 hrs was caused by an initial over-feeding, after which the system recovered; pH increased again, and the feed maintained pH nicely at 4.2. FIG. 6 also shows that yeast propagation proceeds until about 124 hr, and acetic acid remains very low (≦0.2 g/l). FIG. 7 gives the pH profile, it can be seen that pH is kept substantially constant by addition of lignocellulosic hydrolysate from 28 h onwards.

The example shows that propagation on lignocellulosic hydrolysate is possible controlled at a pH (4.2) which is desirable on industrial scale as a means to limit the growth of industrially common contaminating (lactic/acetic acid) bacteria, while feeding with undiluted hydrolysate at a pH (4.3). This hydrolysate pH is in the range (4.0-4.5) that is to be expected after enzymatic hydrolysis. Feeding hydrolysate at this pH abolishes the requirement to add base prior to propagation and thereby lowers yeast propagation costs

In regular propagation processes, operating below the pKa of acetic acid (4.76) while feeding with undiluted hydrolysate would result in severe growth inhibition by acetic acid, rendering the process economically unattractive due to high cellular maintenance energy costs resulting in low biomass yields, and the requirement of long residence times, both resulting in the need for larger aerated fermenters (CAPEX).

### REFERENCES

Verduyn, C., E. Postma, W. A. Scheffers, and J. P. Van Dijken. 1992. Effect of benzoic acid on metabolic fluxes in yeasts: a continuous-culture study on the regulation of respiration and alcoholic fermentation. Yeast 8: 501-517;

Petersson, A. et al; “Fed batch cultivation of Saccharomyces cervisiae on lignocellulosic hydrolysate”. Biotechn. Letters 29, (2) 219-225 (2006);

Andreas, R. to al: “Controlled poliot development unit-scale fed-batch cultivation of yeast on spruce hydrolysate”, Biotechn. progress. 23(2), 351-358 (2007).

1. Process for the aerobic propagation of yeast wherein the yeast is grown in a reactor, comprising the following steps:
a) filling the reactor with carbon source and an initial yeast population,
b) optionally growing the initial yeast population in the reactor in batch mode,
c) measuring the pH in the reactor,
d) adding lignocellulosic hydrolysate to the reactor in fed batch mode at a rate to set the pH in the reactor at a predetermined value,
and
e) after sufficient propagation, isolation of yeast from the reactor.

2. Process according to claim 1, wherein the carbon source is diluted lignocellulosic hydrolysate.

3. Process according to claim 1, wherein the yeast consumes xylose in the lignocelluloic hydrolysate, optionally substantially all xylose.

4. Process according to claim 1, wherein during the process no base needs to be added to the mixture in the reactor.

5. Process according to claim 1, wherein the acetic acid concentration (g/L) is 0.5 g/L or less, optionally 0.2 g/L or less.

6. Process according to claim 1, wherein the lignocellulosic hydrolys ate comprises organic acid.

7. Process according to claim 6, wherein the organic acid is acetic acid.

8. Process according to claim 1, wherein the yeast is capable of metabolizing organic acid, optionally of metabolizing acetic acid.

9. Process according to claim 1, wherein the pH of the mixture in the fed batch reactor in the fed batch mode is kept substantially constant by addition of sufficient lignocellulosic hydrolysate.

10. Process according to claim 1, wherein the concentration of acetic acid in the fed batch reactor is 30 g/l or less.

11. Process according to claim 1, wherein the rate of lignocellulosic hydrolysate fed into the fed batch reactor is 0.10 h⁻¹ or less.

12. Process according to claim 1, wherein the rate of lignocellulosic hydrolysate fed into the fed batch reactor is from 0.01 h⁻¹ to 0.10 h⁻¹.

13. Process according to claim 1, wherein the pH in the reactor in fed batch mode is pH 4 to pH 10, optionally pH 4 to pH 7.

14. Process according to claim 1, wherein the yeast can an-aerobically ferment at least one C6 sugar and at least one C5 sugar.

15. Process according to claim 1, wherein propagation is conducted until at least five generations of growth of the yeast population are realized.

16. Process according to claim 1, wherein propagation is conducted until growth of the yeast population for three or more generations.

17. Process according to claim 16, wherein the propagation is conducted until growth of the yeast population is 5 to 6 generations compared to the initial yeast population.

18. Yeast propagated according to the process according to claim 1.

19. Process for the production of fermentation product wherein a sugar mixture comprising hexose and pentose is anaerobically fermented to fermentation product with a yeast, wherein the yeast is a yeast according to claim 18.

20. Process according to claim 19, wherein the fermentation product is ethanol.
